(19) United States
(12) Patent Application Publication
(10) Pub. No.: US 2026/0265539 A1
(43) Pub. Date: Sep. 10, 2026

(54) WAVELENGTH-CONVERTING ANTIMICROBIAL AND PHOTOBIOMODULATION COATINGS AND MATERIALS

(71) Applicant: SunInLyf Bio Inc., Sleepy Hollow, IL (US)

(72) Inventors: Mike MISKIN, Sleepy Hollow, IL (US); Charles F. HUBER, Lake Forest, IL (US); Allan Brent YORK, Langley (CA)

(21) Appl. No.: 19/556,667

(22) Filed: Mar. 4, 2026

Related U.S. Application Data
(60) Provisional application No. 63/766,499, filed on Mar. 4, 2025.

(51) Int. Cl. C09D 5/14; C09D 7/40; C09D 107/02; C09D 133/02; C09D 175/04

(52) U.S. Cl. CPC C09D 5/14; C09D 7/40; C09D 107/02; C09D 133/02; C09D 175/04

(57) ABSTRACT

Wavelength-converting antimicrobial and photobiomodulation coatings and materials are disclosed. In an example, a wavelength-converting coating includes a coating matrix and at least one wavelength-conversion material incorporated within, dispersed within, or associated with the coating matrix. The wavelength-conversion material is configured to absorb ambient spectral wavelengths of light incident on the wavelength-converting coating and convert at least a portion of the absorbed light into one or more different wavelengths that are re-emitted into a surrounding environment from the wavelength-converting coating outward from a surface to which the wavelength-converting coating is applied.

### PRIORITY CLAIM

This application claims the benefit of U.S. Provisional Application No. 63/766,499, filed Mar. 4, 2025, the entire contents of which are hereby incorporated by reference in their entirety.

### TECHNICAL FIELD

This disclosure relates to wavelength converting antimicrobial and photobiomodulation coatings and materials including but not limited to coatings, paints, dyes, polymers and wearable fabric treatments. More specifically this disclosure relates to water-based and solvent-based coatings, paints, dyes, polymers, and wearable fabric treatments that selectively absorb and convert at least one ambient excitation wavelength of light within the ultraviolet and/or visible spectrum of light while reflecting and/or re-emitting the converted light as at least one of red, near-infrared, infrared “IR”, and/or antimicrobial, antibacterial, antifungal (“antimicrobial”), and/or other wavelengths of light. The disclosure aims to provide thermal regulation, energy efficiency, and aesthetic versatility while maintaining a white, off-white, or custom color appearance to the human eye. Additionally, it reduces the negative effects of selected light wavelength exposure from artificial lighting sources within a surrounding infrastructure, improving visual comfort, minimizing eye strain, and supporting mitochondrial function, circadian rhythm regulation, and improving overall cellular health. This spectral selectivity extends beyond UV and/or blue light absorption to include customized absorption and reflection across the visible and near-infrared spectrum.

### BACKGROUND

Traditional coatings, paints, or fabric treatments utilize pigments such as Titanium Dioxide (TiO₂) to create a white or colored appearance by scattering all visible light wavelengths. While some paints incorporate infrared-reflective materials to reduce heat absorption (e.g., cool roof paints), existing solutions do not provide selective UV and/or blue-light absorption while maintaining a neutral color appearance. These paints and/or coating also do not absorb light from the sun and/or artificial light sources and re-emit beneficial wavelengths of light such as antimicrobial, antibacterial and/or antifungal light.

Current spectral coatings in specialized applications (e.g., smart windows) achieve wavelength selectivity using nanoparticles or quantum dots, but they are not widely adapted for latex paints due to cost, complexity, and manufacturing challenges. The coatings described herein overcome these limitations with a cost-effective, easily manufacturable paint that provides thermal management and selective spectral control.

The role of blue light in LED lighting has created a negative impact on human health. LED technology has revolutionized the lighting industry, becoming the dominant source of illumination in homes, offices, schools, public spaces, and digital screens. Unlike traditional incandescent bulbs, which produce light by heating a filament, LEDs generate light using semiconductor materials that emit photons when an electrical current passes through them. Most white LEDs do not naturally produce white light; instead, they rely on LED chips that emit high-intensity blue light typically around 450-470 nm to excite a phosphor coating, which then emits a spectrum of colors that mix to create white light. This technology is highly energy-efficient, long-lasting, and has been widely adopted in residential lighting, commercial lighting, industrial lighting, streetlights, vehicle headlights, smartphones, TVs, and advertising signage. However, this pervasive shift to LED-based illumination has introduced an unintended consequence: chronic exposure to high-intensity blue wavelengths that can have negative biological effects.

The human eye and cellular health are particularly sensitive to excessive blue light exposure. Blue light penetrates deep into the retina, where it generates oxidative stress and damage to photoreceptor cells, potentially leading to macular degeneration and other vision-related disorders over time. Additionally, blue light suppresses melatonin production, disrupting the circadian rhythm and leading to sleep disturbances and metabolic imbalances. Beyond visual health, emerging research suggests that mitochondria—the energy-producing organelles within our cells—are highly sensitive to specific light wavelengths. Blue light has been found to increase reactive oxygen species (ROS) production in mitochondria, contributing to cellular stress, inflammation, and reduced ATP (energy) production. Since mitochondria play a fundamental role in energy metabolism, brain function, and aging, chronic exposure to artificial blue light may have systemic health implications, affecting neurological health, immune function, and long-term cellular vitality.

Several studies have demonstrated that excessive blue light exposure can lead to oxidative stress and damage in retinal cells:
Photoreceptor Damage: Research indicates that blue light exposure can cause morphological alterations and functional impairments in retinal pigment epithelium (RPE) cells, including reduced phagocytic activity and disrupted secretion of neurotrophic factors, which are essential for photoreceptor health. https://pmc.ncbi.nlm.nih.gov/articles/PMC11685196/
Oxidative Stress Mechanism: Studies have shown that blue light exposure increases the production of reactive oxygen species (ROS) in photoreceptor cells, leading to oxidative stress and potential cell damage. https://pmc.ncbi.nlm.nih.gov/articles/PMC6054877/
Mitochondrial Dysfunction: Exposure to blue light has been observed to cause swelling of mitochondria within retinal ganglion cells, contributing to photoreceptor cell damage. https://www.surveyophthalmol.com/article/S0039-6257%2824%2900079-1/fulltext

These example findings underscore the potential risks associated with the prolonged blue light exposure to the health of humans as it relates to lighting.

Aside from our earthly surroundings and/or nature such as trees, plants, the ground, the sky and water, the infrastructure we live in is predominantly coated and/or painted. There exists a need for reducing blue light exposure from artificial lighting which would have profound benefits for eye health, sleep quality, and cellular well-being. Technologies that selectively filter or absorb excess blue wavelengths without reducing overall lighting efficiency could help mitigate these risks while maintaining high-quality illumination in modern environments.

Traditional paints and coatings are widely used across multiple industries, including residential, commercial, automotive, aerospace, and military applications. While they serve various protective and aesthetic functions, they generally lack wavelength-selective absorption, reflectance and/or conversion with re-emitting capabilities limiting their effectiveness in areas such as health, energy efficiency and other areas.

### Limitations of Existing Coatings & Paint Technologies Across Example Industries

Standard Residential, Commercial and/or Industrial Paints or Coatings (i.e., conventional paints or coatings) are primarily designed for color aesthetics and surface protection, but they do not regulate light wavelengths. This allows for excessive blue light reflection, which has been linked to circadian rhythm disruption, eye strain, and sleep disorders in indoor environments and provides no health benefits at the cellular level.

Energy-Efficient and Infrared-Reflective Coatings—Some coatings incorporate infrared-reflective pigments (e.g., Titanium Dioxide, Aluminum Oxide) to minimize heat absorption in buildings and vehicles. However, they do not provide spectral selectivity across the visible range, leading to inefficient glare control and non-adaptive energy regulation, nor do they provide any health benefits at the cellular level for indoor applications.

Automotive Paints—Vehicle coatings prioritize weather resistance and durability but fail to address glare reduction, eye comfort, or provide any health benefits at the cellular level. Nighttime driving safety could be improved with selective wavelength absorption to reduce excessive blue and UV light reflection from dashboards and interiors.

None of the prior art coatings that exist today are designed to and/or capable of absorbing ambient light from the sun or artificial light sources and re-emitting the light as antibacterial, antimicrobial, antifungal (“antimicrobial”) and/or other biologically beneficial light for health benefits at the cellular level.

Given these limitations, there is a need for a multi-functional paint, dye, and/or coating that selectively absorbs, converts, and re-emits different spectral wavelengths in response to ambient light exposure from sunlight, moonlight, and artificial light to provide antimicrobial, antibacterial, antifungal, and/or other biologically beneficial light for health benefits at the cellular level. The inventions described herein address the shortcomings in existing prior art paint, coating, dye, and wearable fabric treatment technologies.

### SUMMARY

Red, Near-Infrared “NIR”, and/or infrared “IR” light commonly referred to as Red Light Therapy “RLT” and/or photobiomodulation “PBM” improves the efficiency of the cellular respiration process and helps a body produce and use ATP energy more effectively. PBM wavelengths of electromagnetic energy do this by stimulating and/or impacting mitochondria, the powerhouses of the cell. PBM light therapy can increase the number of mitochondria, and also boost their function in the cell to regenerate and/or increase production of ATP and accelerate healing of a wound and/or infection.

Although incandescent lamps were and are inefficient in converting energy to visible light in comparison to LED technology, the incandescent lamps do provide and/or emit healthy wavelengths of visible and/or non-visible Red-IR and/or PBM light that human bodies need and gain health benefits from. The widespread adoption of more power efficient LED technology however uses LED chips that emit unhealthy high-intensity blue light typically around 450-470 nm to excite a phosphor coating, which then emits a spectrum of colors that mix to create white light. Society has unfortunately traded health beneficial lighting for lower cost energy bills and longer life lighting products.

One objective of the disclosure described herein is configured to, but not limited to, providing coatings, paints, dyes, polymers and/or wearable fabric treatments (hereinafter “coating(s)”, “paint(s)”, “dye(s)”, “wearable fabric treatment(s)”, fabric treatment(s), “treatment(s)”) that are configured to absorb at least one ambient excitation wavelength within the ultraviolet and/or visible spectrum spectral wavelengths of light, convert and re-emit the converted spectral wavelengths of light as one or more different spectral wavelengths of light that provide one or more of antimicrobial, antibacterial and/or antifungal (“antimicrobial”) and/or other biologically beneficial PBM wavelengths of light within the visible to non-visible near-red to infrared spectrum for enhancing mitochondrial support and health benefits at the cellular level.

Another objective of the disclosure described herein includes, but is not limited to, manufacturing and providing a coating configured to but not limited to providing a multi-functional coating that absorbs different spectral wavelengths in response to ambient light exposure and converts them into different wavelengths to provide at least one of antimicrobial and/or photobiomodulation wavelengths that reflect back from the surface comprising the coating.

Another objective of the disclosure described herein includes, but is not limited to, manufacturing and providing a coating configured to increase the amount of antimicrobial and/or photobiomodulation wavelengths of light in the air, on nearby surfaces, devices, the coating surface and within the environment of the coating.

Another objective of the disclosure described herein is configured to, but not limited to, providing coatings, paints, dyes, polymers and/or wearable fabric treatments (hereinafter “coating(s)”, “paint(s)”, “dye(s)”, “wearable fabric treatment(s)”, fabric treatment(s), “treatment(s)”) that absorb ambient spectral wavelengths of light, convert and re-emit the converted spectral wavelengths of light as one or more different spectral wavelengths of antimicrobial wavelengths of light within the range of 380 nm-420 nm.

Another objective of the disclosure described herein is configured to, but not limited to, providing coatings, paints, dyes, polymers and/or wearable fabric treatments (hereinafter “coating(s)”, “paint(s)”, “dye(s)”, “wearable fabric treatment(s)”, fabric treatment(s), “treatment(s)”) that absorb ambient spectral wavelengths of light, convert and re-emit the converted spectral wavelengths of light as one or more different spectral wavelengths of PBM light within the range of 580 nm to infrared “IR wavelengths of light.

In certain embodiments, wavelengths commonly associated with photobiomodulation (PBM), including wavelengths within the visible red and near-infrared spectral ranges, may also exhibit antimicrobial or microbial-inhibiting effects under certain conditions. Accordingly, wavelength-conversion materials configured to emit PBM wavelengths may provide both therapeutic photobiomodulation benefits and antimicrobial or microbial-suppression effects within the surrounding environment.

Another objective of the disclosure described herein is configured to, but not limited to, providing water-based and/or solvent-based coatings, paints, dyes, polymers and/or wearable fabric treatments (hereinafter “coating(s)”, “paint(s)”, “dye(s)”, “wearable fabric treatment(s)”, fabric treatment(s), “treatment(s)”) that selectively absorb specific ambient spectral wavelengths while reflecting infrared and other selected wavelengths and/or absorbing UV and/or or blue light and re-emitting the light as antimicrobial wavelengths into the air and/or on surfaces. For avoidance of doubt, the disclosure does not define the term coating as a phosphor coating unless specifically referring to a phosphor coating but the coating may be configured to comprise a phosphor. The disclosure aims to provide thermal regulation, energy efficiency, and aesthetic versatility while maintaining a white, off-white, or custom color appearance. In certain embodiments, the coating is applied to a surface or substrate that is substantially opaque or reflective such that converted wavelengths are emitted outwardly from the coated surface into the surrounding environment rather than transmitted through a transparent optical medium. In certain embodiments, the coating described herein is applied to a surface or substrate such as a wall, ceiling, structural material, device surface, fabric, or other coated article. The coated surface may be substantially opaque, scattering, or reflective such that ambient light incident on the coating is absorbed and spectrally converted by wavelength-conversion materials within the coating and the converted wavelengths are emitted outwardly from the coated surface into the surrounding environment rather than being transmitted through a transparent optical medium.

Another objective of the disclosure described herein is configured to, but not limited to, providing water-based and/or solvent-based coating that reduce the negative effects of selected light wavelength exposure and reflectance including but not limited to blue light typically around 450-470 nm and/or UV light typically within the range of 280-400 nm by absorbing and converting selected wavelengths of light being emitted from ambient artificial lighting sources and/or the sun and re-emitting the wavelengths as PBM light for improving indoor visual comfort, minimizing eye strain, supporting mitochondrial function, circadian rhythm regulation, and improving overall cellular health by providing PBM converted and/or reflected light from surfaces comprising the coatings.

Another objective of the disclosure is to provide coatings that are configured to absorb UV and/or blue light from the sun and/or artificial light sources and re-emit the light as antimicrobial wavelengths into the air and/or on surfaces at one or more wavelengths within the approximate range of 380 nm-420 nm antimicrobial wavelengths including but not limited to one dominant peak wavelength such as 405 nm.

Another objective of the disclosure described herein is configured to, but not limited to, providing a high-performance coating designed to absorb a substantial portion or all visible blue light within the range of 400-495 nm and/or a substantial portion or all UV light within the range of 280-400 nm, while also optionally reflecting red to near-infrared wavelengths (620 nm-2500 nm) and/or Red 600 on up to IR in the range of 1 mm. The formulation ensures high durability, low VOC emissions, and non-toxicity while maintaining ease of application with a roller, spray, or electrostatic deposition. The coating is suitable for residential, commercial, automotive, aerospace, and industrial applications, providing visual comfort, energy efficiency, heat management benefits and improved overall cellular health.

Another objective of the disclosure described herein is configured to, but not limited to, providing water-based and/or solvent-based coating having a spectral selectivity that extends beyond blue light absorption to include customized absorption and reflection across the visible and near-infrared spectrum.

Another objective of the disclosure described herein is configured to, but not limited to, providing a multi-functional coating that selectively reflects different spectral wavelengths in response to ambient light exposure.

Another objective of the disclosure described herein is configured to, but not limited to, providing coating formulations, methods, manufacturing methods and/or processes and product application methods and/or process for applying and/or integrating such coatings that selectively absorb specific spectral wavelengths while reflecting other selected wavelengths and/or providing a wearable coating adaptable to multiple surfaces and fabrics, including water-based latex, oil-based, epoxy, polyurethane paints, and textile-compatible coatings.

Other advantages and aspects of the present invention will become apparent upon reading the following description of the drawings and detailed description of the disclosure.

### BRIEF DESCRIPTION OF THE DRAWINGS

FIG. 1 provides an example schematic drawing according to an example embodiment of the present disclosure.

### DETAILED DESCRIPTION

One embodiment of the disclosure provided herein includes, but is not limited to, manufacturing and providing a multi-functional coating configured to, but not limited to, absorbing different spectral wavelengths in response to ambient light exposure and converting them into different wavelengths to provide at least one of antimicrobial and/or photobiomodulation wavelengths that reflect back from the surface comprising the coating.

Another embodiment of the disclosure described herein includes, but is not limited to, manufacturing and providing a coating configured to increase an amount of antimicrobial and/or photobiomodulation wavelengths of light in the air, on nearby surfaces, devices, the coating surface and within the environment of the coating.

Another embodiment of the disclosure described herein is configured to, but not limited to, providing water-based and/or solvent-based coatings and/or wearables that selectively absorb specific spectral wavelengths of light while reflecting infrared and other selected wavelengths of light. The disclosed coatings are configured to provide health benefits, thermal regulation, energy efficiency, and aesthetic versatility while maintaining a white, off-white, or custom color appearance.

Another embodiment of the disclosure described herein is configured to, but not limited to, providing water-based and/or solvent-based coatings that reduce the negative health effects to vision and mitochondria cells caused by certain and/or select light wavelength exposure and reflectance including but not limited to blue light typically around 450-470 nm primarily being emitted from artificial lighting sources, improving indoor visual comfort, minimizing eye strain, and supporting mitochondrial function, circadian rhythm regulation, and improving overall cellular health.

Another embodiment of the disclosure described herein is configured to, but not limited to, providing water-based and/or solvent-based coatings having a spectral selectivity that extends beyond blue light absorption to include customized absorption and reflection across the visible and near-infrared spectrum.

Another embodiment of the disclosure described herein is configured to, but not limited to, providing a high-performance coating designed to absorb a substantial portion of selected high-energy visible wavelengths of blue light within the range of 400-495 nm and/or UV light typically within the range of 280-400 nm by while optionally reflecting red to near-infrared wavelengths of light within the range of 620-2500 nm, and in some cases above 2500 nm. The formulation ensures high durability, low VOC emissions, and non-toxicity while maintaining ease of application with a roller, spray, or electrostatic deposition. The coating is suitable for residential, commercial, automotive, aerospace, and industrial applications, providing visual comfort, energy efficiency, heat management benefits and improving overall cellular health.

Another embodiment of the disclosure described herein is configured to, but not limited to, providing a multi-functional coating that selectively absorbs and/or reflects different spectral wavelengths in response to ambient light exposure according to the light emissions of the sun and/or artificial light emissions at specific wavelengths of emission, and/or selectable reflectance of such light sources improving overall cellular health.

Another embodiment of the disclosure is to provide coatings that are configured to absorb white, UV, and/or blue light from the sun and/or artificial light sources and re-emit the light as antimicrobial wavelengths of light into the air and/or on surfaces within the approximate range of 380 nm-420 nm antimicrobial wavelengths including but not limited to one dominant peak wavelength such as 405 nm wavelengths including but not limited to 405 nm.

In certain embodiments, the coating is applied to a surface or substrate that is substantially opaque or reflective such that converted wavelengths are emitted outwardly from the coated surface into the surrounding environment rather than transmitted through a transparent optical medium.

In certain embodiments, the coating described herein is applied to a surface or substrate such as a wall, ceiling, structural material, device surface, fabric, or other coated article. The coated surface may be substantially opaque, scattering, or reflective such that ambient light incident on the coating is absorbed and spectrally converted by wavelength-conversion materials within the coating and the converted wavelengths are emitted outwardly from the coated surface into the surrounding environment rather than being transmitted through a transparent optical medium.

Another embodiment of the disclosure described herein is configured to, but not limited to, providing a multi-functional polymer composition that selectively absorbs and/or reflects different spectral wavelengths in response to ambient light exposure according to the light emissions of the sun and/or artificial light emissions at specific wavelengths of emission, and/or selectable reflectance of such light sources improving overall cellular health.

Another embodiment of the disclosure described herein is configured to, but not limited to, providing multi-functional coatings with integrated photochromic, thermochromic, quantum dot, and/or low-emissivity materials to create a next-generation coating.

Another embodiment of the disclosure described herein is configured to, but not limited to, providing a coating, coating formulations, methods, manufacturing methods and/or processes and product application methods and/or process for applying and/or integrating such coatings that selectively absorb specific spectral wavelengths while reflecting other wavelengths including but not limited to infrared and/or other selected wavelengths and/or providing a wearable coating adaptable to multiple surfaces and fabrics, including water-based latex, oil-based, epoxy, polyurethane paints, and textile-compatible coatings.

Another embodiment of the disclosure described herein is configured to, but not limited to, providing a multi-purpose, wavelength-selective coating and/or fabric coating designed to absorb all visible wavelengths of blue light within the range of 400-495 nm, selectively manage red to near-infrared wavelengths of light within the range of 620-2500 nm, and dynamically respond to temperature changes. The formulation provides high durability, low VOC emissions, and non-toxicity, while maintaining ease of application with roller, spray, or electrostatic deposition. The coating may be configured to be available in a standard blue absorption and IR reflectance coating, or in blue, UV absorption with IR reflectance capabilities or any combination thereof or any other combination wavelengths being absorbed and/or reflected for interior and/or exterior surfaces of building, transportation vehicles for land, sea, air and/or space, robotics and/or wearables. Additionally, this coating provides aerospace benefits by maintaining microbial suppression and surface sanitation in aerospace cabins and high-contact aerospace surfaces.

Another embodiment of the disclosure is configured to comprise a multi-functional coating that selectively absorbs and/or reflects different spectral wavelengths in response to ambient light exposure by utilizing organic dyes or specialized pigments, quantum dots and/or other spectral conversion materials and/or chemicals to remove specific wavelengths, including but not limited to blue (400 nm-495 nm), green (495 nm-570 nm), red (620 nm-750 nm), and near-infrared to IR spectrum (750 nm-2500 nm+). The coating may be configured to comprise Customizable Spectral Selectivity to control specific wavelength absorption or reflection based on environmental needs, industry applications, and other capabilities and/or requirements. The coating may be configured to provide and comprise a formulation configured to be integrated into textiles to create uniforms and gear that respond to varying lighting conditions and thermal exposure. The coating may be configured to comprise Reduction of Mitochondrial Oxidative Stress by being configured to comprise a Selective Wavelength Filtering Mechanism that reduces oxidative damage at the cellular level, helping to prevent neurological degeneration, cognitive decline, and metabolic stress. The coating may be configured to comprise Reflection of Infrared and Selected Wavelengths by being configured to comprise NIR and/or IR reflective pigments such as Titanium Dioxide (TiO₂), Aluminum Oxide (Al₂O₃), or Boron Nitride (BN) to enhance reflectivity and reduce heat absorption. The coating may be configured to comprise Applicability Across Various Paint Types & Finishes and be configured to provide and/or be compatible with matte, semi-gloss, high-gloss, satin, and textured finishes, as well as indoor and outdoor applications. The coating may be configured to comprise Low-VOC and Non-Toxic Composition to ensure compliance with environmental and safety regulations for household, commercial, industrial, and defense applications. The coating may be configured to selectively absorb and/or reflect different spectral wavelengths in response to ambient light exposure according to environmental light emissions of the sun at specific wavelengths of emission at different times of the day under different climates and/or weather conditions, the light emissions of the moon at different times of the day under different climates and/or weather conditions, light emissions and/or reflectance in water, and/or artificial light emissions.

Another embodiment of the disclosure comprises providing a finished product configured to be semi-coated and/or substantially completely coated, infused, impregnated, and/or doped with the coating including but not limited to building materials, clothing, electronic equipment, military equipment, transportation vehicles for land, water, air and or space such as cars, trucks, ships, planes or other finished products.

The coating may be made and configured to absorb various spectral emissions including but not limited to blue light and reflect wavelengths of NIR to IR light and may be configured to but not be limited to comprising the following recipe, technical specification and example composition for manufacturing and/or applying the coating as outlined below in items 1-6 under “Example: Spectral Selective Coating Specifications and Composition 1.A”

### Example: Spectral Selective Coating Specifications and Composition 1.A

### 1. General Description

A high-performance coating configured to absorb a substantial portion of selected ultraviolet and/or high-energy visible wavelengths while reflecting and/or re-emitting selected red, near-infrared, and/or infrared wavelengths. The formulation ensures durability, low VOC emissions, and ease of application by roller, spray, or electrostatic deposition. The coating is suitable for residential, commercial, automotive, aerospace, and industrial applications, providing visual comfort, biological lighting management, energy efficiency, and heat management benefits.

### 2. Composition

### 2.1 Base Composition (for Various Coating Types)

Binder System (Variable Based on Coating Type)—40-50% by weight
   Water-Based Latex & Acrylic Coatings: Acrylic Emulsion or Styrene-Acrylic Binder
   Oil-Based & Alkyd Coatings: Alkyd-Modified Acrylic or Waterborne Alkyd Binder
   Epoxy-Based Coatings: Epoxy-Modified Acrylic or Epoxy Resin
   Polyurethane & Urethane Coatings: Acrylic-Polyurethane Hybrid Binder
   High-Temperature & Specialty Coatings: Silicone-Modified Acrylic Binder
   Function: Provides durability, adhesion, and flexibility across coating types

### 2.2 Functional Additives

Spectrally Selective Absorbing Additives—Effective concentration
   Configured to absorb at least one selected excitation wavelength within the ultraviolet and/or visible spectrum for wavelength conversion and/or spectral management.
Near-Infrared (NIR) Reflective Pigments (Optional)—Effective concentration
   Examples: Titanium Dioxide (TiO₂), Aluminum Oxide (Al₂O₃), Boron Nitride (BN), ceramic-based reflectors.
   Function: Enhances red and infrared reflectivity while maintaining desired color appearance.
Luminescent Materials (optional)—Effective Concentration
   Rare-earth-doped phosphors, aluminate-based phosphors, silicate-based phosphors, perovskite-based materials, quantum dots, or other wavelength-tunable emitters.
Dispersants & Surfactants—1-3%
Matting Agents—1-5%
Biocides & Preservatives—Effective concentration

### 3. Physical & Optical Properties

Color Appearance: Customizable
Light Absorption & Conversion: At least one excitation wavelength within the ultraviolet and/or visible spectrum
Light Reflection and/or Re-Emission: At least one wavelength within the visible, near-infrared, and/or infrared spectrum
Gloss Level: Matte to Gloss
Density: Approximately 1.2-1.4 g/cm³
VOC Content: ≤5 g/L
Application Method: Roller, spray, electrostatic deposition
Durability: Comparable to commercial-grade coatings

### 4. Performance & Durability

Weather Resistance
UV Stability
Chemical Resistance
Adhesion & Flexibility

### Optional Thermal Management via Infrared Reflectivity

This example formulation as described above under “Example: Spectral Selective Coating Specifications and Composition A” provides selective blue-light absorption and infrared reflectivity to improve health factors, visual comfort, energy efficiency, and heat management. Unlike conventional coatings, this solution provides enhanced health by reducing and/or eliminating blue light and/or UV reflection from artificial light sources, reduces eye strain and circadian disruption caused by excessive blue light exposure, improved overall cellular health, while also enhancing thermal regulation in buildings, vehicles, and industrial applications.

Another embodiment of the disclosure comprises a coating formulated for high-efficiency antimicrobial emission, where the composition is designed to absorb ultraviolet and/or high-energy visible wavelengths from ambient light sources including but not limited to the sun and/or artificial indoor and outdoor lights inside building structures and/or transportation vehicles, then convert and re-emit the converted wavelengths as light within the approximate range of 380 nm-420 nm antimicrobial wavelengths including but not limited one dominant peak wavelength such as 405 nm, providing continuous microbial suppression on treated surfaces. The coating incorporates phosphor-based or quantum-dot-enhanced additives, optimized for maximum spectral conversion efficiency, ensuring that a significant portion of absorbed blue light is effectively down-converted into one or more of the antimicrobial wavelengths within the range of 380 nm-420 nm. This conversion process is achieved through a wavelength-selective matrix embedded within a polymeric or latex-based binder, allowing the coating to be applied to walls, ceilings, and high-contact surfaces in hospitals, food processing areas, and public spaces. Additionally, the coating may integrate a reflective substrate to enhance the outward emission of 405 nm light and/or other wavelengths within the range of 380 nm-420 nm, reducing internal energy losses and increasing surface coverage of antimicrobial radiation. This embodiment further supports customization in gloss levels, pigmentation, and durability enhancements, ensuring compatibility with various environmental conditions, surface types, and regulatory safety standards. In certain configurations, the formulation may include optional red/infrared reflective materials, allowing for dual-function applications that combine antimicrobial efficacy with thermal regulation for energy efficiency. This coating provides long-term, passive antimicrobial functionality without requiring active power sources or chemical disinfectants, making it an ideal solution for continuous protection in healthcare, residential, and commercial settings.

The coating may be made and configured to absorb various spectral emissions including but not limited to UV, blue light within the range of 400 nm-495 nm and down-convert the wavelengths to one or more antimicrobial wavelengths of light such as 405 nm light which is then reflected back out from the coating. The coating may be applied to a reflective surface including but not limited to another coated surface such as a white wall which then provides for more reflectivity of the converted antimicrobial wavelengths of light back out from the surface.

The coating may be configured to but not be limited to comprising the following example recipe, technical specification and example composition for manufacturing and/or applying the coating as outlined below in items 1-7 under “Example: Spectral Selective Antimicrobial Paint & Coating Specifications and Composition 1.B”

### Example: Spectral Selective Antimicrobial Paint & Coating Specifications and Composition 1.B

### 1. General Description

A wavelength-conversion antimicrobial coating configured to absorb selected excitation wavelengths within the ultraviolet and/or visible spectrum and re-emit light within antimicrobial wavelength ranges including wavelengths near 405 nm, thereby providing passive microbial suppression of treated surfaces.

### 2. Composition

### 2.1 Base Composition—40-50% Binder System

Acrylic, Latex, Epoxy, Polyurethane, or Hybrid Systems
   2.2 Functional Additives—Effective concentrations
Luminescent Down-Conversion Materials
Optional Reflective Substrate Layer
UV Stabilizers
Adhesion Promoters
Water & Chemical Resistance Enhancers

### 3. Functional Characteristics

Passive antimicrobial emission upon excitation
Optional infrared reflection
Indoor and outdoor compatibility

### Protective Layer Embodiments:

In certain embodiments, the wavelength-conversion coating layer may be covered by one or more optically transmissive protective layers configured to enhance environmental durability while maintaining excitation and emission functionality. Such layers may reduce oxygen ingress, moisture exposure, ultraviolet exposure, abrasion, and other degradation mechanisms. The system may comprise a multilayer architecture including a substrate, wavelength-conversion layer, and protective sealant layer.

In certain embodiments, the wavelength-conversion coating layer may be applied directly to a substrate surface and may optionally be covered by one or more overlying protective layers configured to enhance environmental durability while maintaining optical functionality. Such protective layers may be substantially optically transmissive at one or more excitation wavelengths and one or more emission wavelengths associated with the wavelength-conversion material, such that light transmission to and from the wavelength-conversion layer is not materially impaired. The optional protective layer may be configured to reduce or inhibit environmental degradation mechanisms including, but not limited to, oxygen ingress, moisture exposure, ultraviolet exposure, thermal cycling, abrasion, chemical contact, and physical wear. In some embodiments, the protective layer may function as a barrier layer to reduce exposure of the wavelength-conversion material to oxygen and/or moisture, thereby enhancing operational lifespan and performance stability over time. The protective layer may comprise any suitable optically transmissive material including, without limitation, polymeric materials, crosslinked coatings, inorganic-organic hybrid coatings, laminates, sealants, sol-gel derived materials, or multilayer structures. The protective layer may be applied sequentially, co-applied, laminated, cured in situ, or otherwise formed over the wavelength-conversion coating layer using spraying, rolling, brushing, dipping, curtain coating, printing, vapor deposition, lamination, or other surface application techniques. In certain embodiments, the system may comprise a multilayer architecture including a substrate, the wavelength-conversion coating layer, and an overlying protective sealant layer, wherein the protective sealant layer provides environmental barrier properties while preserving the intended optical excitation and emission characteristics of the wavelength-conversion material.

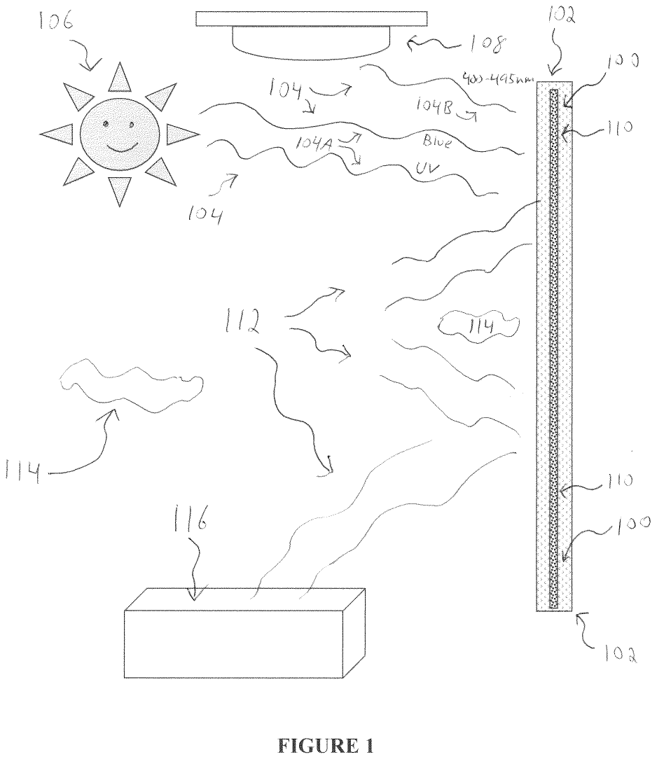

FIG. 1 shows and describes one example embodiment of a coating 100 according to the disclosure described herein. The coating 100 is configured to be added to a surface 102 and absorb light including but not limited to UV and/or Blue wavelengths of light 104 from a natural source such as the sun 106 that emits natural light 104A or a lighting device 108 that emits artificial light 104B. The coating 100 is configured to comprise light absorbing, reflecting and/or conversion materials 110 integrated within it such as phosphors, quantum dots and/or other materials capable of absorbing, converting and/or re-emitting different wavelengths of light from in comparison to the wavelengths of light 104A and 104B that was originally absorbed from the sun 106 or lighting device 108. The coating 100 is configured to receive and/or absorb the available wavelengths of light 104 such as UV, Blue and/or other wavelengths from the sun 106 and/or lighting device 108 and re-emit 405 nm antimicrobial light 112 and/or other antimicrobial wavelengths back from the coating into the air 114 and/or onto any surface 116. The coating 100 as described herein is configured to but not limited to provide a high-performance coating designed to absorb a substantial portion of selected high-energy visible wavelengths of blue light within the range of 400-495 nm and/or UV light typically within the range of 280-400 nm while optionally reflecting red to near-infrared wavelengths of light within the range of 620-2500 nm, and in some cases above 2500 nm. The formulation ensures high durability, low VOC emissions, and non-toxicity while maintaining ease of application with a roller, spray, or electrostatic deposition. The coating is suitable for residential, commercial, automotive, aerospace, and industrial applications, providing visual comfort, energy efficiency, heat management benefits and improving overall cellular health. Another embodiment of the disclosure comprises a coating formulated for high-efficiency antimicrobial emission, where the composition is designed to absorb ultraviolet and/or high-energy visible wavelengths from ambient light sources including but not limited to the sun and/or artificial indoor and outdoor lights inside building structures and/or transportation vehicles, then convert and re-emit the converted wavelengths as light within the approximate range of 380 nm-420 nm antimicrobial wavelengths, providing continuous microbial suppression on treated surfaces. The coating incorporates phosphor-based or quantum-dot-enhanced additives, optimized for maximum spectral conversion efficiency, ensuring that a significant portion of absorbed blue light is effectively down-converted into the antimicrobial 405 nm wavelength. This conversion process is achieved through a wavelength-selective matrix embedded within a polymeric or latex-based binder, allowing the coating to be applied to walls, ceilings, and high-contact surfaces in hospitals, food processing areas, and public spaces. Additionally, the coating may integrate a reflective substrate to enhance the outward emission of 405 nm light, reducing internal energy losses and increasing surface coverage of antimicrobial radiation. This embodiment further supports customization in gloss levels, pigmentation, and durability enhancements, ensuring compatibility with various environmental conditions, surface types, and regulatory safety standards. In certain configurations, the formulation may include optional red/infrared reflective materials, allowing for dual-function applications that combine antimicrobial efficacy with thermal regulation for energy efficiency. This coating provides long-term, passive antimicrobial functionality without requiring active power sources or chemical disinfectants, making it an ideal solution for continuous protection in healthcare, residential, and commercial settings.

The various structural features, material compositions, wavelength ranges, emission characteristics, encapsulation approaches, optical management structures, and application environments described herein may be implemented individually or in any combination or sub-combination, and the disclosure expressly contemplates embodiments directed to any such combinations and sub-combinations, whether or not explicitly illustrated or described in a single embodiment

As used herein, the term “wavelength-conversion material” refers to any material, compound, particle, or structure configured to absorb electromagnetic radiation at one wavelength or range of wavelengths and re-emit radiation at one or more different wavelengths. Non-limiting examples include phosphors, quantum dots, dyes, pigments, luminescent materials, and combinations thereof.

As used herein, the term “ambient spectral wavelengths” refers to electromagnetic radiation present in an environment, including radiation originating from natural sources such as sunlight and artificial sources such as lighting devices.

While the foregoing there has set forth embodiments of the disclosure, it is to be understood that the present disclosure may be embodied in other forms without departing from the spirit or central characteristics thereof. The present embodiments, therefore, are to be considered in all respects as illustrative and not restrictive, and the disclosure is not to be limited to the details given herein. While specific embodiments have been illustrated and described, numerous modifications come to mind without significantly departing from the characteristics of the disclosure and the scope of protection is only limited by the scope of the accompanying claims.

The invention is claimed as follows:

1. A wavelength-converting coating comprising:
a coating matrix; and
at least one wavelength-conversion material incorporated within, dispersed within, or associated with the coating matrix,
wherein the at least one wavelength-conversion material is configured to absorb ambient spectral wavelengths of light incident on the wavelength-converting coating and convert at least a portion of the absorbed light into one or more different wavelengths that are re-emitted into a surrounding environment from the wavelength-converting coating outward from a surface to which the wavelength-converting coating is applied.

2. The wavelength-converting coating of claim 1, wherein the ambient spectral wavelengths include ultraviolet wavelengths, visible wavelengths, or combinations thereof.

3. The wavelength-converting coating of claim 1, wherein the at least one wavelength-conversion material comprises at least one of phosphors, quantum dots, dyes, pigments, or luminescent materials.

4. The wavelength-converting coating of claim 1, wherein the wavelength-converting coating is configured to absorb ultraviolet and/or blue wavelengths and convert and re-emit the absorbed light as different wavelengths.

5. The wavelength-converting coating of claim 1, further comprising reflective pigments configured to reflect infrared wavelengths.

6. The wavelength-converting coating of claim 1, wherein the coating matrix comprises a binder compatible with water-based coatings, solvent-based coatings, latex paints, epoxy coatings, polyurethane coatings, or combinations thereof.

7. The wavelength-converting coating of claim 1, wherein the wavelength-converting coating is configured to maintain a white, off-white, or colored appearance while converting spectral wavelengths.

8. A wavelength-converting coating comprising:
a coating matrix; and
at least one wavelength-conversion material configured to absorb ambient light and convert and re-emit the absorbed light as photobiomodulation (PBM) wavelengths within a visible red to near-infrared spectral range.

9. The wavelength-converting coating of claim 8, wherein the PBM wavelengths include visible red wavelengths.

10. The wavelength-converting coating of claim 8, wherein the PBM wavelengths include near-infrared wavelengths.

11. The wavelength-converting coating of claim 8, wherein the at least one wavelength-conversion material comprises phosphors, quantum dots, dyes, pigments, or combinations thereof.

12. The wavelength-converting coating of claim 8, wherein the wavelength-converting coating is configured to convert ultraviolet and/or blue wavelengths into photobiomodulation wavelengths.

13. The wavelength-converting coating of claim 8, wherein the wavelength-converting coating is configured to emit photobiomodulation wavelengths into an environment surrounding a coated surface.

14. The wavelength-converting coating of claim 8, wherein the wavelength-converting coating is configured for application to residential, commercial, industrial, transportation, or wearable surfaces.

15. A wavelength-converting antimicrobial coating comprising:
a coating matrix; and
at least one wavelength-conversion material configured to absorb ambient spectral wavelengths of light and convert and re-emit the absorbed light as antimicrobial wavelengths.

16. The wavelength-converting antimicrobial coating of claim 15, wherein the antimicrobial wavelengths include wavelengths within an approximate range of 380 nm to 420 nm.

17. The wavelength-converting antimicrobial coating of claim 15, wherein the antimicrobial wavelengths include a dominant peak wavelength within a range of 405 nm to 420 nm.

18. The wavelength-converting antimicrobial coating of claim 15, wherein the at least one wavelength-conversion material comprises phosphors, quantum dots, dyes, pigments, or luminescent materials.

19. The wavelength-converting antimicrobial coating of claim 15, wherein the wavelength-converting antimicrobial coating is configured to convert ultraviolet and/or blue wavelengths into the antimicrobial wavelengths.

20. The wavelength-converting antimicrobial coating of claim 15, wherein the antimicrobial wavelengths are emitted into air and onto surfaces surrounding a coated structure.

21. The wavelength-converting antimicrobial coating of claim 15, wherein the wavelength-converting antimicrobial coating is configured to provide passive antimicrobial emission when exposed to ambient light from the sun or artificial lighting.

22. A wavelength-converting polymer composition comprising:
a polymer matrix; and
at least one wavelength-conversion material incorporated within the polymer matrix and configured to absorb ambient spectral wavelengths of light, convert the absorbed light into one or more different wavelengths, and re-emit the converted wavelengths.

23. The wavelength-converting polymer composition of claim 22, wherein the polymer matrix comprises a thermoplastic material.

24. The wavelength-converting polymer composition of claim 22, wherein the polymer matrix comprises polycarbonate, acrylic, polypropylene, polyethylene, or combinations thereof.

25. The wavelength-converting polymer composition of claim 22, wherein the at least one wavelength-conversion material comprises phosphors, quantum dots, dyes, pigments, or combinations thereof.

26. The wavelength-converting polymer composition of claim 22, wherein the wavelength-converting polymer composition is configured for injection-molded or extruded plastic products.

27. A coated article comprising:
a substrate; and
a coating applied to the substrate,
wherein the coating comprises a coating matrix and at least one wavelength-conversion material configured to absorb ambient spectral wavelengths of light, convert the absorbed light into one or more different wavelengths, and re-emit the converted wavelengths outward from a coated surface of the substrate into a surrounding environment.

28. The coated article of claim 27, wherein the substrate comprises a building surface.

29. The coated article of claim 27, wherein the substrate comprises a transportation surface, device surface, structural surface, or fabric material.

30. The coated article of claim 27, wherein the coating is configured to convert ultraviolet and/or visible wavelengths into photobiomodulation wavelengths or antimicrobial wavelengths emitted from the coated surface into the surrounding environment.
